(19) United States
(12) Patent Application Publication
(10) Pub. No.: US 2013/0165474 A1
(43) Pub. Date: Jun. 27, 2013

(54) COMPOUNDS THAT INHIBIT TAU PHOSPHORYLATION

(71) Applicant: Travis Dunckley, Scottsdale, AZ (US)

(72) Inventor: Travis Dunckley, Scottsdale, AZ (US)

(21) Appl. No.: 13/817,340

(22) PCT Filed: Aug. 17, 2011
(86) PCT No.: PCT/US11/48132

§ 371 Date: Feb. 22, 2013

Related U.S. Application Data
(60) Provisional application No. 61/374,324, filed on Aug. 17, 2010.
(60) Provisional application No. 61/391,238, filed on Oct. 8, 2010.
(60) Provisional application No. 61/452,409, filed on Mar. 14, 2011.

(51) Int. Cl. A61K 31/437

(52) U.S. Cl. CPC A61K 31/437
USPC 514/300

(57) ABSTRACT

The present invention provides methods and compositions for enhancing working memory impaired in a tau pathological condition associated with AD or Down's syndrome.

### CROSS REFERENCE

This application claims the priority of U.S. provisional application with application No. 61/452,409, filed on Mar. 14, 2011; U.S. provisional application with application No. 61/374,324, filed on Aug. 17, 2010; U.S. provisional applications with application No. 61/391,235, filed on Oct. 8, 2010, which are hereby incorporated by reference in their entirety.

### STATEMENT REGARDING FEDERALLY SPONSORED RESEARCH

The US government retains certain rights in this invention as provided by the terms of Grant Numbers R21AG029576 and K01AG024079 awarded by the National Institutes of Health.

### FIELD OF THE INVENTION

The present invention is related to methods and compositions for treatment of neurodegenerative diseases where phosphorylated tau protein is deposited within neurons in the form of neurofibrillary tangles (NFTs). Specifically, the invention is related to working memory enhancement using tau phosphorylation inhibitors.

### BACKGROUND OF THE INVENTION

Disorders of the brain are serious medical conditions causing disability and diminished quality of life. Alzheimer's disease (AD) is the most common cause of disabling memory and thinking problems in older persons. Alzheimer's disease (AD) is a neurodegenerative disease associated with progressive memory loss and cognitive dysfunction.

### (i) Alzheimer's Disease and Tau Protein

Clinically, Alzheimer's disease is characterized by gradual but progressive declines in memory, language skills, the ability to recognize objects or familiar faces, the ability to perform routine tasks, judgment, and reasoning. Associated features commonly include agitation, paranoid delusions, sleepiness, aggressive behaviors, and wandering. In its most severe form, patients may be confused, bed-ridden, unable to control their bladder or bowel functions, or swallow.

Neuropathologically, AD is characterized by the accumulation of (1) neuritic plaques, the major component of which is the amyloid-B peptide (Aβ), and (2) neurofibrillary tangles (NFT), the major component of which is the hyper-phosphorylated form of the protein tau. While the etiology leading to the development of AD has not been clearly resolved, it was observed that hyperphosphorylation of the tau protein can result in the self-assembly of tangles of paired helical filaments and straight filaments, and thus leads to the pathogenesis of tauopathies as a contributing factor.

In fact, in all neurodegenerative diseases in which tau pathology has been observed, the tau is abnormally phosphorylated. In adult human brain, there are six major isoforms of tau generated by alternative mRNA splicing. Elevated levels of phosphorylated tau correlate with the presence of dynamic microtubules during periods of high plasticity in the developing mammalian brain. The phosphorylation of tau at specific sites is the predominant mechanism by which tau function is regulated. The longest form of adult human brain tau has 80 Ser or Thr residues and 5 Tyr residues; therefore, almost 20% of the molecule has the potential to be phosphorylated. The majority of sites on tau that are phosphorylated are Ser/Thr-Pro sites, Ser and Thr sites not followed by Pro residues are also phosphorylated. Dynamic, site-specific phosphorylation of tau is essential for its proper functioning. Inappropriate phosphorylation of tau, which leads to tau dysfunction, results in decreased cell viability.

In vitro, tau is a substrate for many protein kinases; however, only a few are considered to be good candidates for bona fide in vivo tau kinases. For example, one likely tau kinase is glycogen synthase kinase 3β (GSK3β). There are often conflicting reports and theories regarding whether a kinase is an in vivo Tau kinase because of intertwined regulation mechanisms. Using cyclin-dependent kinase 5 (Cdk5), an in vitro tau kinase, as an example, it was observed that, the subcellular localization and physiological versus pathological conditions of Cdk5 activator, p35, is tightly regulated, which adds layers of complication to the relationship between Cdk5 and Tau phosphorylation. Further, a tau kinase may indirectly regulate the kinases and phosphatases that act on tau, which adds even more complexity to Tau phosphorylation. For example, Cdk5 phosphorylates two protein phosphatase 1 (PP1) inhibitors, I-1 and I-2. Because PP1 can dephosphorylate tau, activation of I-1 by Cdk5 phosphorylation should enhance tau phosphorylation. However, phosphorylation of I-2 by Cdk5 prevents it from inhibiting PP1, which counteracts the effect of I-1 phosphorylation, and this might result in a shift towards tau dephosphorylation. Another study demonstrated that the inhibition of Cdk5 leads to PP1 activation and subsequent dephosphorylation and activation of GSK3β, the net result being increased phosphorylation of tau. In addition, there are a plural number of kinases that phosphorylate tau in vivo. The known ones that are likely to be tau in vivo kinase include GSK3β, PKA (cAMP-dependent protein kinase), MARK (microtubule-affinity-regulating kinase), and some others. Although there is good evidence that Cdk5 regulates tau phosphorylation in vivo, it remains to be determined whether this is predominantly a direct or indirect effect. Kinase effect on tau, in vivo or not, a direct or indirect, predominant or not, complicates the selection of a kinase as a therapeutic target.

Further, the phosphorylation sites of tau are relevant to tau pathology. MARK selectively phosphorylates a KXGS motif, which is present in each microtubule-binding repeat of tau, as well as other microtubule-associated proteins. MARK probably phosphorylates these epitopes more efficiently in situ than do other protein kinases, because tau is phosphorylated at KXGS motifs in vivo (Ser262 being the most prominently phosphorylated KXGS motif).

Phosphorylation of the KXGS motifs within the microtubule-binding repeats of tau strongly reduces the binding of tau to microtubules in vitro and probably in vivo. Although in vitro studies showed that phosphorylation of Ser262 alone is sufficient to attenuate significantly the ability of tau to bind microtubules in vitro, in situ phosphorylation of two or more KXGS motifs (especially Ser262 and Ser356) is required to decrease microtubule binding and facilitate the formation of cell processes.

Phosphorylation of Thr231 by GSK3β also plays a significant role in regulating tau-microtubule interactions; however, Ser235 must be phosphorylated first to get efficient phosphorylation of Thr231. Phosphorylation of Thr231 greatly diminishes the ability of tau to bind microtubules in situ. By contrast, phosphorylation of tau at Ser396 and/or Ser404 does not significantly affect the ability of tau to bind to microtubules. Pseudophosphorylation (changing Ser to Glu) of Ser396, Ser404 and Ser422 generates tau that is more fibrillogenic. However, not all tau phosphorylation events that lead to decreased microtubule binding contribute to the development of tau pathology. For example, although phosphorylation of Ser262 (and Ser214) on tau decreases the affinity of tau for microtubules, these phosphorylation events inhibit tau polymerization into filaments. Further, there may be multiple events that synergize with abnormal phosphorylation events to drive tau polymerization in brain affected by AD. The fact that tau protein that has been cleaved by caspase is more fibrillogenic than full-length tau supports this hypothesis. The protein kinases contributing to the pathological phosphorylation of tau in AD and other neurodegenerative diseases remain elusive. Further, between Aβ production and its down stream tau phosphorylation event, numerous hypotheses have been put forth; however, the exact role that tau hyperphosphorylation plays in pathogenic processes remains unclear.

### (ii) DYRK1A and its Inhibitors

DYRK1A (Dual-specificity tyrosine phosphorylation-regulated kinase) has been shown to be important for phosphorylation of tau protein on multiple sites in several cell models. The DYRK1A is a dual-specificity protein kinase that catalyses the phosphorylation of serine and threonine residues in its substrates as well as the autophosphorylation on a tyrosine residue within an activation loop. The human DYRK1A gene was identified as a Down syndrome candidate gene, because of its localization in the Down syndrome critical region on human chromosome 21. Overexpression of DYRK1A has been proposed to be a significant contributor to the underlying neurodevelopmental abnormalities associated with Down syndrome. Transgenic animals overexpressing DYRK1A show marked cognitive deficits and impairment in hippocampal dependent memory tasks. Studies in cell culture models and transgenic models of Down syndrome that over-expressed DYRK1A implicate the role of DYRK1A kinase in the generation of both amyloid and tau pathologies associated with the late onset Alzheimer's disease (LOAD) that is uniformly observed in Down Syndrome. However, the possible DYRK1A genetic association to AD suggested by Kimura et al using tagging SNPs located in haplotype blocks in the DYRK1A gene was not able to be repeated in a different population (Vazquez-Higuer J L et al, BMC Med Genet. 10, 129 (2009)).

The identified DYRK1A inhibitors, as research tools, include but are not limited to purvalanol, DMAT (2-dimethylamino-4,5,6,7-tetrabromo-1H-benzimidazole), TBB (4,5,6,7-tetrabromo-1H-benzotriazole), pyrazolidine-3,5-diones 18 and 21, TG003 (a benzothiazole derivative), INDY (a benzothiazole derivative), EGCG (epigallocatechin-gallate) and harmine. Those compounds, originally designed to target other protein kinases, were later uncovered as fairly efficient inhibitors of DYRK1A. Pyrazolidinedione compounds inhibit DYRK1A autophosphorylation with IC50 values from 0.6-2.5 μM. INDY is a benzothiazol inhibitor of DYRK1A with IC50 values around 0.24 μM.

According to one study, AD afflicts about 10% of those over the age of 65 and almost half of those over the age of 85. The age-specific prevalence of dementia increases from 1.5% by the age of 60 years to 40% in nonagenarians. An estimated 4 million Americans have AD. By the year 2030 approximately 1 in every 80 persons in the U.S. will have AD.

From the time of diagnosis, people with AD survive about half as long as those of similar age without dementia. Medicare costs for beneficiaries with AD were $91 billion in 2005 and may increase to as much as $160 billion in 2010. By contributing to other problems (e.g., inanition and infections), it is considered the fourth leading cause of death in the United States. A therapy of the illness helps to decelerate the patients' cognitive decline, prolongs a self-determined, independent life and, thus, would reduce the immense care-giving expenses. Therefore, finding a treatment that could delay the onset of and/or alleviate the AD condition is in great need.

### SUMMARY OF THE INVENTION

One aspect of the present invention provides a pharmaceutical composition which comprises at least one pharmaceutically acceptable carrier and at least one compound selected from the group consisting of the following structure:

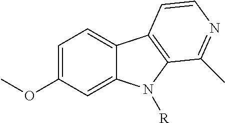

The R in the above structure is selected from the group consisting of H, halo, —C₁-C₆ alkyl, aryl, —C₃-C₇ cycloalkyl, and -3- to 10-membered heterocycle, harmine, harmol, harmane, norharmane, harmaline, and 9-ethyl harmine. One preferred pharmaceutical composition comprises 9-ethyl harmine and at least one pharmaceutically acceptable carrier. Alternatively, said pharmaceutical composition comprises harmol and at least one pharmaceutically acceptable carrier. Similarly, said pharmaceutical composition may comprise harmane and at least one pharmaceutically acceptable carrier. And said pharmaceutical composition may comprise harmine and at least one pharmaceutically acceptable carrier. In a further embodiment, said pharmaceutical comprises at least one pharmaceutically acceptable carrier and at least one compound selected from the group consisting of 9-ethyl harmine, harmol, harmane and harmine.

Another aspect of the invention provides a method of treating a disorder that includes phosphorylation of a serine or threonine residue of tau protein represented by SEQ ID NO. 1. Said method comprises administering a therapeutically effective dose of a pharmaceutical composition comprising a compound selected from the group consisting of a structure as follows:

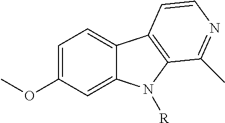

The R in the above structure is selected from the group consisting of H, halo, —C₁-C₆ alkyl, aryl, —C₃-C₇ cycloalkyl, and -3- to 10-membered heterocycle, harmine, harmol, harmane, norharmane, harmaline, and 9-ethyl harmine. In one embodiment of this aspect, the disorder treated by said method is Alzheimer's disease. In another embodiment of this aspect, the disorder treated by said method is Down's syndrome. Said method targets the serine or threonine residue that is selected from the group consisting of serine-262, threonine-231, and serine-396 of tau protein as represented by SEQ ID NO. 1. Specifically, in said method, the pharmaceutical composition comprises 9-ethyl harmine and at least one pharmaceutically acceptable carrier. In another embodiment of this aspect, the composition in said method comprises harmol and at least one pharmaceutically acceptable carrier. In another embodiment of this aspect, said composition may comprise harmine and at least one pharmaceutically acceptable carrier. Alternatively, the composition in said method comprises harmane and at least one pharmaceutically acceptable carrier. In a further embodiment of this aspect, the composition in said method comprises at least one pharmaceutically acceptable carrier and at least one compound selected from the group consisting of 9-ethyl harmine, harmol, harmane and harmine.

Yet another aspect of the present invention provides a method of enhancing the working memory of a subject comprising the step of administering a therapeutically active dose of a pharmaceutical composition comprising a compound selected from the group consisting of the structure as follows:

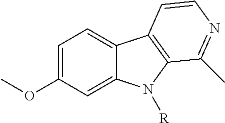

The R in the above structure is selected from the group consisting of H, halo, —C₁-C₆ alkyl, aryl, —C₃-C₇ cycloalkyl, and -3- to 10-membered heterocycle, harmine, harmol, harmane, norharmane, harmaline, and 9-ethyl harmine to the subject. In one preferred embodiment of this aspect, the pharmaceutical composition in said method comprises 9-ethyl harmine and at least one pharmaceutically acceptable carrier. Alternatively, the composition in said method comprises harmol and at least one pharmaceutically acceptable carrier. In another alternative embodiment of this aspect, the composition in said method comprises harmane and at least one pharmaceutically acceptable carrier. In another embodiment of this aspect, the composition in said method may comprise harmine and at least one pharmaceutically acceptable carrier. As a further potential embodiment of this aspect, the composition in said method comprises at least one pharmaceutically acceptable carrier and at least one compound selected from the group consisting of 9-ethyl harmine, harmol, harmane and harmine. In one preferred embodiment of this aspect, the subject treated using said method has Alzheimer's disease. Alternatively, the subject treated using said method has Down's syndrome.

Other aspects and iterations of the invention are described in more detail below.

### BRIEF DESCRIPTION OF THE FIGURES

FIG. 1 illustrates that reduced DYRK1A expression affects tau phosphorylation at multiple sites in vitro. Silencing of DYRK1A was confirmed with anti-DYRK1A antibody (top panel). Percent control values represent the average of three independent siRNA transfections and westerns. NS refers to the non-silencing control. 12E8 refers to the dual phosphorylation epitope pS262/pS356.

FIG. 2 illustrates that harmine affects tau phosphorylation. (A) is the toxicity profile of harmine against the H4 neuroglioma cell line. The harmine IC₅₀ for viability was 12 μM. (B) showed the dose-dependent reduction of total tau phosphorylation and all three phosphorylated forms of tau in H4-tau cells treated with harmine at the indicated concentrations. % control values represent the amount of the respective tau forms present following treatment with harmine at different concentration. (C) showed the results for moclobemide, an MAO-A selective antagonist, did not affect tau phosphorylation following treatment with even the highest 500 μM concentration.

FIG. 3 illustrates that multiple β-carboline derivatives affect levels of total tau and phosphorylated tau. Each indicated compound was tested at the concentrations shown below each compound name. % control values represent the effect seen at the highest concentration tested for each compound.

FIG. 4 illustrates that harmine inhibits the DYRK1A catalyzed direct phosphorylation of tau protein on serine 396. In (A) are results of an in vitro phosphorylation assay utilizing recombinant DYRK1A and tau proteins. A doublet pS396 tau phosphorylation is observed only in the presence of tau, DYRK1A, and ATP. In (B), harmine potently inhibits the direct phosphorylation of tau protein by DYRK1A with an IC₅₀ of 0.7 μM.

FIG. 5 illustrates that structurally distinct β-carboline derivatives inhibit DYRK1A-dependent pS396 tau phosphorylation with varying affinities. Shown are in vitro phosphorylation results for all compounds in this study. The compounds tested are indicated above the respective western results for each compound. The concentrations (in μM) are indicated at the top of the first panel and are the same for each compound tested. The IC₅₀ values calculated from these assays are indicated in the right column, next to the western results for each compound.

FIG. 6 depicts the Delayed-match-to-sample asymmetrical 3-choice task for evaluating the spatial working memory and short-term memory retention of the rats treated with harmine.

FIG. 7 depicts the Morris water maze for evaluating the spatial reference memory of the rats treated with harmine.

FIG. 8 depicts the Visible platform task for evaluating the motor and visual competence of the rats treated with harmine.

FIG. 9 presents the regression analysis indicated that in Harmine-high treated animals, test squad was a significant predictor of mean escape latency across trials 1-6 on the visible platform task (β=−11.567, SE=2.751, p=0.006, R²=0.75). The inset shows that in Harmine-low and vehicle-treated groups, test squad is not predictive of mean escape latency (β=−0.08, SE=1.702, p=0.96 NS, R²<0.01).

FIG. 10 presents the Mean±SE total errors on the DMS asymmetrical 3 choice task for trials 2-6, testing block 4 (Days 8-9). Combined Harmine-high and -low treated animals made fewer errors relative to vehicle-treated animals (F_{1,24}=5.036, P=0.03).

FIG. 11 presents (A) Mean±SE distance swam to the platform for MM days 1-3, trials 1-6. There were no Harmine treatment main effects (F_{2,23}=1.497; P=0.24 NS). (B) Mean±SE % distance swam in previously platformed (NE) vs. opposite (SW) quadrant on the probe trial. A higher percent distance was spent in the previously platformed vs. the opposite quadrant (quadrant main effect: F_{1,24}=149.187; P<0.0001). This was in the absence of a Treatment×Quadrant interaction, indicating that all groups localized to the previously platformed quadrant.

### DETAILED DESCRIPTION OF THE INVENTION

The present invention provides methods and compositions treating Tauopathies, a class of neurodegenerative diseases where tau protein is deposited within neurons in the form of neurofibrillary tangles (NFTs), which include AD and Down Syndrome.

### (I) Pharmaceutical Composition

One aspect of the invention provides using harmine and derivatives thereof to enhance working memory which is impaired under tau phosphorylation pathological condition, including AD and Down's syndrome.

Harmine, a β-carboline alkaloid, has long been known as a potent inhibitor of monoamine oxidase A (IC₅₀=5 nm). Harmine is produced by divergent plant species, including the South American vine Banisteriopsis caapi and the mideastern shrub Peganum harmala (Syrian rue). Banisteriopsis is a component of hoasca, a hallucinogenic brew of plant extracts used in shamanic rituals and South American sects for its visionary effects. The monoamine oxidase-inhibiting activity of harmine blocks the first pass metabolism of dimethyltryptamine by monoamine oxidase A and thereby allows the oral ingestion of this natural hallucinogenic. The family of β-carboline alkaloids, characterized by a core indole structure and a pyridine ring, affects multiple central nervous system targets. These include the 5-hydroxytryptamine receptor substypes 5-HT₂ and 5-HT_{1A}, the NMDA receptor, monoamine oxidase (MAO-A) and dopaminergic signaling pathways.

One aspect of the invention provides a pharmaceutical composition comprising a compound with the following structure:

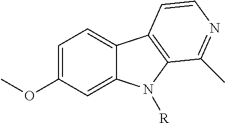

R may be H, halo, —C₁-C₆ alkyl, aryl, —C₃-C₇ cycloalkyl, 3- or 10-membered heterocycle, any of which may be unsubstituted or substituted with one or more of the following: -halo, —C₁-C₆ alkyl, —O—(C₁-C₆ alkyl), —OH, —CN, —COOR′, —OC(O)R′, NHR′, N(R′)₂, —NHC(O)R′ or —C(O)NHR′, wherein R′ may be —H or —C₁-C₆ alkyl.

A —C₁-C₆ alkyl group includes any straight or branched, saturated or unsaturated, substituted or unsubstituted hydrocarbon comprising between one and six carbon atoms. Examples of —C₁-C₆ alkyl groups include, but are not limited to methyl, ethyl, propyl, isopropyl, butyl, sec-butyl, tert-butyl, pentyl, isopentyl, neopentyl, hexyl, isohexyl, neohexyl, ethylenyl, propylenyl, 1-butenyl, 2-butenyl, 1-pentenyl, 2-pentenyl, 1-hexenyl, 2-hexenyl, 3-hexenyl, acetylenyl, pentynyl, 1-butynyl, 2-butynyl, 1-pentynyl, 2-pentynyl, 1-hexynyl, 2-hexynyl and 3-hexynyl groups. Substituted —C₁-C₆ alkyl groups may include any applicable chemical moieties. Examples of groups that may be substituted onto any of the above listed —C₁-C₆ alkyl groups include but are not limited to the following examples: halo, —C₁-C₆ alkyl, —O—(C₁-C₆ alkyl), —OH, —CN, —COOR′, —OC(O)R′, —NHR′, N(R′)₂, —NHC(O)R′ or —C(O)NHR′ groups. The groups denoted R′ above may be —H or any —C₁-C₆ alkyl.

An aryl group includes any unsubstituted or substituted phenyl or napthyl group. Examples of groups that may be substituted onto ay aryl group include, but are not limited to: halo, —C₁-C₆ alkyl, —O—(C₁-C₆ alkyl), —OH, —CN, —COOR′, —OC(O)R′, NHR′, N(R′)₂, —NHC(O), R′, or —C(O)NEtR′. The group denoted R′ may be —H or any —C₁-C₆ alkyl.

A C₃-C₇ cycloalkyl group includes any 3-, 4-, 5-, 6-, or 7-membered substituted or unsubstituted non-aromatic carbocyclic ring. Examples of C₃-C₇ cycloalkyl groups include, but are not limited to, cyclopropyl, cyclobutyl, cyclopentyl, cyclopentadienyl, cyclohexyl, cyclohexenyl, cycloheptyl, cycloheptanyl, 1,3-cyclohexadienyl, -1,4-cyclohexadienyl, -1,3-cycloheptadienyl, and -1,3,5-cycloheptatrienyl groups. Examples of groups that may be substituted onto C₃-C₇ cycloalkyl groups include, but are not limited to: -halo, —C₁-C₆ alkyl, —O—(C₁-C₆ alkyl), —OH, —CN, —COOR′, —OC(O)R′, NHR′, N(R′)₂, —NHC(O)R′ or —C(O)NHR′ groups. The groups denoted R′ above include an —H or any unsubstituted —C₁-C₆ alkyl, examples of which are listed above. Halo groups include any halogen. Examples include but are not limited to —F, —Cl, —Br, or —I.

A heterocycle may be any optionally substituted saturated, unsaturated or aromatic cyclic moiety wherein said cyclic moiety is interrupted by at least one heteroatom selected from oxygen (O), sulfur (S) or nitrogen (N). Heterocycles may be monocyclic or polycyclic rings. For example, suitable substituents include halogen, halogenated C₁-C₆ alkyl, halogenated C₁-C₆ alkoxy, amino, amidino, amido, azido, cyano, guanidino, hydroxyl, nitro, nitroso, urea, OS(O)₂R; OS(O)₂OR, S(O)₂OR S(O)₀₋₂R, C(O)OR wherein R may be H, C₁-C₆ alkyl, aryl or 3 to 10 membered heterocycle) OP(O)OR₁OR₂, P(O)OR₁OR₂, SO₂NR₁R₂, NR₁SO₂R₂C(R₁)NR₂C(R₁)NOR₂, R₁ and R₂ may be independently H, C₁-C₆ alkyl, aryl or 3 to membered heterocycle), NR₁C(O)R₂, NR₁C(O)OR₂, NR₃C(O)NR₂R₁, C(O)NR₁R₂, OC(O)NR₁R₂. For these groups, R₁, R₂ and R₃ are each independently selected from H, C₁-C₆ alkyl, aryl or 3 to 10 membered heterocycle or R₁ and R₂ are taken together with the atoms to which they are attached to form a 3 to 10 membered heterocycle.

Possible substituents of heterocycle groups include halogen (Br, Cl, I or F), cyano, nitro, oxo, amino, C₁₋₄ alkyl (e.g., CH₃, C₂H₅, isopropyl) C₁₋₄ alkoxy (e.g., OCH₃, OC₂H₅), halogenated C₁₋₄ alkyl (e.g., CF₃, CHF₂), halogenated C₁₋₄ alkoxy (e.g., OCF₃, OC₂F₅), COOH, COO—C₁₋₄ alkyl, CO—C₁₋₄ alkyl, C₁₋₄ alkyl —S— (e.g., CH₃S, C₂H₅S), halogenated C₁₋₄ alkyl —S— (e.g., CF₃S, C₂F₅S), benzyloxy, and pyrazolyl.

Examples of heterocycles include but are not limited to azepinyl, aziridinyl, azetyl, azetidinyl, diazepinyl, dithiadiazinyl, dioxazepinyl, dioxolanyl, dithiazolyl, furanyl, isooxazolyl, isothiazolyl, imidazolyl, morpholinyl, morpholino, oxetanyl, oxadiazolyl, oxiranyl, oxazinyl, oxazolyl, piperazinyl, pyrazinyl, pyridazinyl, pyrimidinyl, piperidyl, piperidino, pyridyl, pyranyl, pyrazolyl, pyrrolyl, pyrrolidinyl, thiatriazolyl, tetrazolyl, thiadiazolyl, triazolyl, thiazolyl, thienyl, tetrazinyl, thiadiazinyl, triazinyl, thiazinyl, thiopyranyl furoisoxazolyl, imidazothiazolyl, thienoisothiazolyl, thienothiazolyl, imidazopyrazolyl, cyclopentapyrazolyl, pyrrolopyrrolyl, thienothienyl, thiadiazolopyrimidinyl, thiazolothiazinyl, thiazolopyrimidinyl, thiazolopyridinyl, oxazolopyrimidinyl, oxazolopyridyl, benzoxazolyl, benzisothiazolyl, benzothiazolyl, imidazopyrazinyl, purinyl, pyrazolopyrimidinyl, imidazopyridinyl, benzimidazolyl, indazolyl, benzoxathiolyl, benzodioxolyl, benzodithiolyl, indolizinyl, indolinyl, isoindolinyl, furopyrimidinyl, furopyridyl, benzofuranyl, isobenzofuranyl, thienopyrimidinyl, thienapyridyl, benzothienyl, cyclopentaoxazinyl, cyclopentafuranyl, benzoxazinyl, benzothiazinyl, quinazolinyl, naphthyridinyl, quinolinyl, isoquinolinyl, benzopyranyl, pyridopyridazinyl and pyridopyrimidinyl groups.

The disclosed compound and its intermediates may exist in different tautomeric forms. Tautomers include any structural isomers of different energies that have a low energy barrier to interconversion. One example is proton tautomers (prototropic tautomers.) In this example, the interconversions occur via the migration of a proton. Examples of prototropic tautomers include, but are not limited to keto-enol and imine-enamine isomerizations. In another example illustrated graphically below, proton migration between the 1-position and 3-position nitrogen atoms of the benzimidazole ring may occur. As a result, Formulas Ia and Ib are tautomeric forms of each other:

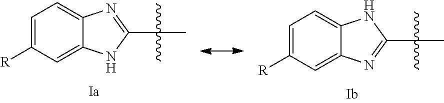

The disclosed compound further encompasses any other physiochemical or stereochemical form that the disclosed compound may assume. Such forms include diastereomers, racemates, isolated enantiomers, hydrated forms, solvated forms, or any other known or yet to be disclosed crystalline, polymorphic crystalline, or amorphous form. Amorphous forms lack a distinguishable crystal lattice and therefore lack an orderly arrangement of structural units. Many pharmaceutical compounds have amorphous forms. Methods of generating such chemical forms will be well known by one with skill in the art.

The disclosed compound also encompasses structures indicated in Table 1 below and their equivalents and derivatives.

TABLE 1

| Compound | Structure | IC₅₀ |
|---|---|---|
| harmine | 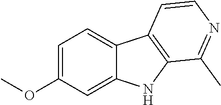 | 12 μM |
| harmol | 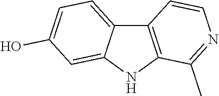 | 18 μM |
| harmane | 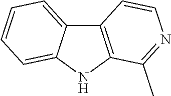 | 32 μM |
| norharmane | 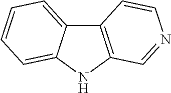 [SMILES: c1ccc2c(c1)[nH]c1cnccc12] | 95 μM |
| harmaline | 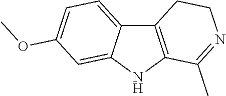 [SMILES: COc1ccc2c3c([nH]c2c1)C(C)=NCC3] | 56 μM |
| 9-ethyl harmine | 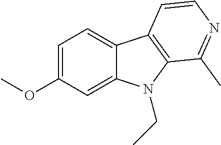 | 9 μM |

The invention encompasses pharmaceutical compositions that include one or more beta-carboline derivatives as an ingredient. In one embodiment of the invention, the pharmaceutical composition comprises harmine, and at least one pharmaceutically acceptable carrier. In another embodiment of the invention, the pharmaceutical composition comprises 9-ethyl harmine, and at least one pharmaceutically acceptable carrier. In yet another embodiment of the invention, the pharmaceutical composition comprises harmol, and at least one pharmaceutically acceptable carrier. Alternatively, in one embodiment of the invention, the pharmaceutical composition comprises harmane and at least one pharmaceutically acceptable carrier. Still in another embodiment, the pharmaceutical composition comprises at least one pharmaceutically acceptable carrier and at least one compound selected from the group consisting of harmine, 9-ethyl harmine, harmol and harmane.

Such pharmaceutical compositions may take any physical form necessary depending on a number of factors including the desired method of administration and the physicochemical and stereochemical form taken by the compound or pharmaceutically acceptable salts of the compound. Such physical forms include a solid, liquid, gas, sol, gel, aerosol, or any other physical form now known or yet to be disclosed.

The concept of a pharmaceutical composition including the disclosed compound also encompasses the disclosed compound or a pharmaceutically acceptable salt thereof with or without any other additive. The physical form of the invention may affect the route of administration and one skilled in the art would know to choose a route of administration that takes into consideration both the physical form of the compound and the disorder to be treated. Pharmaceutical compositions that include the disclosed compound may be prepared using methodology well known in the pharmaceutical art. A pharmaceutical composition that includes the disclosed compound may include a second effective compound of a distinct chemical formula from the disclosed compound. This second effective compound may have the same or a similar molecular target as the target or it may act upstream or downstream of the molecular target of the disclosed compound with regard to one or more biochemical pathways.

Pharmaceutical compositions including the disclosed compound include materials capable of modifying the physical form of a dosage unit. In one nonlimiting example, the composition includes a material that forms a coating that contains the compound. Materials that may be used in a coating, include, for example, sugar, shellac, gelatin, or any other inert coating agent.

Pharmaceutical compositions including the disclosed compound may be prepared as a gas or aerosol. Aerosols encompass a variety of systems including colloids and pressurized packages. Delivery of a composition in this form may include propulsion of a pharmaceutical composition including the disclosed compound through use of liquefied gas or other compressed gas or by a suitable pump system. Aerosols may be delivered in single phase, bi-phasic, or tri-phasic systems.

In some aspects of the invention, the pharmaceutical composition including the disclosed compound is in the form of a solvate. Such solvates are produced by the dissolution of the disclosed compound in a pharmaceutically acceptable solvent. Pharmaceutically acceptable solvents include any mixtures of more than one solvent. Such solvents may include pyridine, chloroform, propan-1-ol, ethyl oleate, ethyl lactate, ethylene oxide, water, ethanol, and any other solvent that delivers a sufficient quantity of the disclosed compound to treat the condition without serious complications arising from the use of the solvent in a majority of patients.

Pharmaceutical compositions that include the disclosed compound may also include a pharmaceutically acceptable carrier. Carriers include any substance that may be administered with the disclosed compound with the intended purpose of facilitating, assisting, or helping the administration or other delivery of the compound. Carriers include any liquid, solid, semisolid, gel, aerosol or anything else that may be combined with the disclosed compound to aid in its administration. Examples include diluents, adjuvants, excipients, water, oils (including petroleum, animal, vegetable or synthetic oils.) Such carriers include particulates such as a tablet or powder, liquids such as oral syrup or injectable liquid, and inhalable aerosols. Further examples include saline, gum acacia, gelatin, starch paste, talc, keratin, colloidal silica, and urea. Such carriers may further include binders such as ethyl cellulose, carboxymethylcellulose, microcrystalline cellulose, or gelatin; excipients such as starch, lactose or dextrins; disintegrating agents such as alginic acid, sodium alginate, Primogel, and corn starch; lubricants such as magnesium stearate or Sterotex; glidants such as colloidal silicon dioxide; sweetening agents such as sucrose or saccharin, a flavoring agent such as peppermint, methyl salicylate or orange flavoring, or coloring agents. Further examples of carriers include polyethylene glycol, cyclodextrin, oils, or any other similar liquid carrier that may be formulated into a capsule. Still further examples of carriers include sterile diluents such as water for injection, saline solution, physiological saline, Ringer's solution, isotonic sodium chloride, fixed oils such as synthetic mono or digylcerides, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose, thickening agents, lubricating agents, and coloring agents.

The pharmaceutical composition including the disclosed compound may take any of a number of formulations depending on the physicochemical form of the composition and the type of administration. Such forms include solutions, suspensions, emulsions, tablets, pills, pellets, capsules, capsules including liquids, powders, sustained-release formulations, directed release formulations, lyophylates, suppositories, emulsions, aerosols, sprays, granules, powders, syrups, elixirs, or any other formulation now known or yet to be disclosed. Additional examples of suitable pharmaceutical carriers are well known in the art.

Methods of administration include, but are not limited to, oral administration and parenteral administration. Parenteral administration includes, but is not limited to intradermal, intramuscular, intraperitoneal, intravenous, subcutaneous, intranasal, epidural, sublingual, intramsal, intracerebral, iratraventricular, intrathecal, intravaginal, transdermal, rectal, by inhalation, or topically to the ears, nose, eyes, or skin. Other methods of administration include but are not limited to infusion techniques including infusion or bolus injection, by absorption through epithelial or mucocutaneous linings such as oral mucosa, rectal and intestinal mucosa. Compositions for parenteral administration may be enclosed in ampoule, a disposable syringe or a multiple-dose vial made of glass, plastic or other material.

Administration may be systemic or local. Local administration is administration of the disclosed compound to the area in need of treatment. Examples include local infusion during surgery; topical application, by local injection; by a catheter; by a suppository; or by an implant. Administration may be by direct injection into the central nervous system by any suitable route, including intraventricular and intrathecal injection. Intraventricular injection can be facilitated by an intraventricular catheter, for example, attached to a reservoir, such as an Ommaya reservoir. Pulmonary administration may be achieved by any of a number of methods known in the art. Examples include use of an inhaler or nebulizer, formulation with an aerosolizing agent, or via perfusion in a fluorocarbon or synthetic pulmonary surfactant. The disclosed compound may be delivered in the context of a vesicle such as a liposome or any other natural or synthetic vesicle.

A pharmaceutical composition formulated to be administered by injection may be prepared by dissolving the disclosed compound with water so as to form a solution. In addition, a surfactant may be added to facilitate the formation of a homogeneous solution or suspension. Surfactants include any complex capable of non-covalent interaction with the disclosed compound so as to facilitate dissolution or homogeneous suspension of the compound.

Pharmaceutical compositions including the disclosed compound may be prepared in a form that facilitates topical or transdermal administration. Such preparations may be in the form of a solution, emulsion, ointment, gel base, transdermal patch or iontophoresis device. Examples of bases used in such compositions include opetrolatum, lanolin, polyethylene glycols, beeswax, mineral oil, diluents such as water and alcohol, and emulsifiers and stabilizers, thickening agents, or any other suitable base now known or yet to be disclosed.

Determination of an effective amount of the disclosed compound is within the capability of those skilled in the art, especially in light of the detailed disclosure provided herein. The effective amount of a pharmaceutical composition used to affect a particular purpose as well as its toxicity, excretion, and overall tolerance may be determined in cell cultures or experimental animals by pharmaceutical and toxicological procedures either known now by those skilled in the art or by any similar method yet to be disclosed. One example is the determination of the IC₅₀ (half maximal inhibitory concentration) of the pharmaceutical composition in vitro in cell lines or target molecules. Another example is the determination of the LD₅₀ (lethal dose causing death in 50% of the tested animals) of the pharmaceutical composition in experimental animals. The exact techniques used in determining an effective amount will depend on factors such as the type and physical/chemical properties of the pharmaceutical composition, the property being tested, and whether the test is to be performed in vitro or in vivo. The determination of an effective amount of a pharmaceutical composition will be well known to one of skill in the art who will use data obtained from any tests in making that determination. Determination of an effective amount of disclosed compound for administration also includes the determination of an effective therapeutic amount and a pharmaceutically acceptable dose, including the formulation of an effective dose range for use in vivo, including in humans.

The toxicity and therapeutic efficacy of a pharmaceutical composition may be determined by standard pharmaceutical procedures in cell cultures or animals. Examples include the determination of the IC₅₀ (the half maximal inhibitory concentration) and the LD₅₀ (lethal dose causing death in 50% of the tested animals) for a subject compound. The data obtained from these cell culture assays and animal studies can be used in formulating a range of dosage for use in human. The dosage may vary depending upon the dosage form employed and the route of administration utilized.

The effective amount of the disclosed compound to results in the slowing of expansion of the cancer cells would preferably result in a concentration at or near the target tissue that is effective in slowing cellular expansion in neoplastic cells, but have minimal effects on non-neoplastic cells, including non-neoplastic cells exposed to radiation or recognized chemotherapeutic chemical agents. Concentrations that produce these effects can be determined using, for example, apoptosis markers such as the apoptotic index and/or caspase activities either in vitro or in vivo.

Treatment of a condition is the practice of any method, process, or procedure with the intent of halting, inhibiting, slowing or reversing the progression of a disease, disorder or condition, substantially ameliorating clinical symptoms of a disease disorder or condition, or substantially preventing the appearance of clinical symptoms of a disease, disorder or condition, up to and including returning the diseased entity to its condition prior to the development of the disease. Generally, the effectiveness of treatment is determined by comparing treated groups with non-treated groups.

The addition of a therapeutically effective amount of the disclosed compound encompasses any method of dosing of a compound. Dosing of the disclosed compound may include single or multiple administrations of any of a number of pharmaceutical compositions that include the disclosed compound as an active ingredient. Examples include a single administration of a slow release composition, a course of treatment involving several treatments on a regular or irregular basis, multiple administrations for a period of time until a diminution of the disease state is achieved, preventative treatments applied prior to the instigation of symptoms, or any other dosing regimen known in the art or yet to be disclosed that one skilled in the art would recognize as a potentially effective regimen. A final dosing regimen including the regularity of and mode of administration will be dependent on any of a number of factors including but not limited to the subject being treated; the severity of the condition; the manner of administration, the stage of disease development, the presence of one or more other conditions such as pregnancy, infancy, or the presence of one or more additional diseases; or any other factor now known or yet to be disclosed that affects the choice of the mode of administration, the dose to be administered and the time period over which the dose is administered.

Pharmaceutical compositions that include the disclosed compound may be administered prior to, concurrently with, or after administration of a second pharmaceutical composition that may or may not include the compound. If the compositions are administered concurrently, they are administered within one minute of each other. If not administered concurrently, the second pharmaceutical composition may be administered a period of one or more minutes, hours, days, weeks, or months before or after the pharmaceutical composition that includes the compound Alternatively, a combination of pharmaceutical compositions may be cyclically administered. Cycling therapy involves the administration of one or more pharmaceutical compositions for a period of time, followed by the administration of one or more different pharmaceutical compositions for a period of time and repeating this sequential administration, in order to reduce the development of resistance to one or more of the compositions, to avoid or reduce the side effects of one or more of the compositions, and/or to improve the efficacy of the treatment.

The invention further encompasses kits that facilitate the administration of the disclosed compound to a diseased entity. An example of such a kit includes one or more unit dosages of the compound. The unit dosage would be enclosed in a preferably sterile container and would be comprised of the disclosed compound and a pharmaceutically acceptable carrier. In another aspect, the unit dosage would comprise one or more lyophilates of the compound. In this aspect of the invention, the kit may include another preferably sterile container enclosing a solution capable of dissolving the lyophilate. However, such a solution need not be included in the kit and may be obtained separately from the lyophilate. In another aspect, the kit may include one or more devices used in administrating the unit dosages or a pharmaceutical composition to be used in combination with the compound. Examples of such devices include, but are not limited to, a syringe, a drip bag, a patch or an enema. In some aspects of the invention, the device comprises the container that encloses the unit dosage.

Pharmaceutical compositions including the disclosed compound may be used in methods of treating memory loss or enhancing memory. Such methods involve the administration of an effective amount of a pharmaceutical composition that includes the disclosed compound and/or a pharmaceutically acceptable salt thereof to a mammal.

### (II) Method of Enhancing Working Memory Relevant to AD

Another aspect of the invention provides methods of enhancing working memory relevant to AD in a subject. The subjects to the provided method include but are not limited to mammals (particularly humans) as well as other mammals of economic or social importance, including those of an endangered status. Further examples include livestock or other animals generally bred for human consumption and domesticated companion animals.

Although long-term memory deficits are the hallmark of AD, deficits in short-term memory of information as well as higher level deficits result in AD patients related to the diminished ability to coordinate multiple tasks or to inhibit irrelevant information. Short-term memory is also referred to as working memory, primary memory, immediate memory, operant memory, or provisional memory. Short-term/working memory tasks are those that require the goal-oriented active monitoring or manipulation of information or behaviors in the face of interfering processes and distractions. Working memory can be divided into separate systems for retaining location information and object information (colors, shapes), which are commonly referred to as spatial working memory (SWM) and visual (or object) working memory (VWM), respectively. In one embodiment, the method provided enhances the short-term memory in the AD patient such that the impairments in dual-task performance, inhibitory ability, and set-shifting ability are alleviated. In one embodiment, the method provided enhances the short-term memory in the AD patient such that the ability to remember information over a brief period of time (in the order of seconds), and the ability to actively hold information in the mind needed to do complex tasks such as reasoning, comprehension and learning is improved.

The methods of enhancing working memory associated to AD patient may comprise the step of testing the working memory capacity during and after the treatment. The working memory capacity can be tested by a variety of tasks. With animals, such as rats, mazes are commonly used to determine whether different treatments or conditions affect learning and memory in rats. For example, the Multiple T-maze, a complex maze made of many T-junctions, or the Y-maze with three identical arms, can be used to answer questions of place versus response learning and cognitive maps; can be used to answer questions of place versus response learning and cognitive maps. The radial arm maze, in general, having a center platform with eight, twelve, or sixteen spokes radiating out from a central core, can be used for testing short-term memory. To test this, a single food pellet is placed at the end of each arm. A rat is placed on the central platform. The rat visits each arm and eats the pellet. To successfully complete the maze, the rat must go down each arm only once. He must use short-term memory and spatial cues to remember which arms he's already visited. If a rat goes down an arm twice, this counts as an error. The rats might be given particular drugs or treatment conditions to see if these impair or enhance short-term memory. In one embodiment, the subject may be administered a pharmaceutical composition comprising at lease one compound selected from the group consisting of 9-ethyl harmine, harmol, harmane and harmine.

Working memory can also be tested using the Morris water maze. In general, the Morris water maze is a large round tub of opaque water with two small hidden platforms located 1-2 cm under the water's surface. The rat is placed on a start platform. The rat swims around until it finds the other platform to stand on. External cues, such as patterns or the standing researcher, are placed around the pool in the same spot every time to help the rat learn where the end platform is. The researcher measures how long it takes for a rat to find hidden platform, by changing or moving and using different spatial cues. The Morris water maze tests the spatial learning, cognitive maps and memory. The rats under the Morris water maze test may be given particular drugs or treatment conditions to see if these impair or enhance short-term memory. In one embodiment, the subject may be administered a pharmaceutical composition comprising at lease one compound selected from the group consisting of 9-ethyl harmine, harmol, harmane and harmine.

Other methods of evaluating spatial and visual working memory include Delayed-Match to Sample (DMS) asymmetrical 3-choice task, which is illustrated in detail in Example section. The visible platform task was used to confirm that animals have the ability to perform the procedural components of water-escape maze testing, including the visual and motoric capacities necessary to swim towards and climb onto a platform.

For human subjects, nonlimiting example of various neurologic exams in a patient with a suspected dementia include “Wechlser” Memory Scales test, Halstead-Reitan Battery, Trails A and B, Boston Naming Test, Benton Visual Retention Test or Graham-Kendall Memory-for-Designs, Rey-Osterrieth Complex Figure Test, Controlled Oral Word Association Test, tests for left visual neglect, Folstein's Mini-Mental State Exam (MMSE). One or more of these tests may be taken before, during or after the period of treatment characterized by administering a pharmaceutical composition comprising at least one compound selected from the group consisting of 9-ethyl harmine, harmol, harmane and harmine.

### EXAMPLES

The following examples illustrate certain aspects of the invention. It is to be understood, however, that these examples are provided by way of illustration only, and nothing therein should be deemed a limitation upon the overall scope of the invention.

### Example 1

This Example demonstrates that Harmine and other β-carboline derivatives reduced tau phosphorylation.

Materials and Methods:

siRNA Transfection:

4RON tau overexpressing H4 neuroglioma cells were maintained in Dulbecco's Modified Eagle Medium supplemented with 10% fetal bovine serum, 1% penicillin-streptomycin, geneticin (0.25 mg/ml), and 2 mM L-Glutamine. Cells were maintained by splitting 1:10 at 90% confluency. Prior to any experimentation, cells were 70-75% confluent to ensure cells were in their active growth phase. To test effects of DYRK1A knockdown on tau phosphorylation, cells were transfected with DYRK1A siRNA. Prior to treating cells with DYRK1A siRNA, siRNA was first complexed with siLentfect lipid transfection reagent (Bio-Rad, Hercules, Calif.) and reduced serum medium using a 6 well plate format. The final effective siRNA molarity used was 22.85 nM per well. Cells were grown for 96 hours at 37° C., 5% CO2. Cell lysates were prepared using the Complete Lysis-M, EDTA-free kit (Roche Applied Science, Indianapolis, Ind.) and total protein concentration was quantified using the BCA protein assay (Pierce, Rockford, Ill.). Westerns for the multiple forms of tau were performed as described below.

Western Blotting:

For all cell-based experiments, including siRNA treatments and compound treatments, cells were treated for 96 hours at 37° C., with 5% CO₂. Cell lysates were then prepared using the Complete Lysis-M, EDTA-free kit (Roche Applied Science) and quantified using the BCA protein assay (Pierce). Protein from lysates (30 μg total protein per lane) was separated on SDS-PAGE gels and transferred to nitrocellulose membrane. Membranes were blocked in 5% blocking solution for one hour at room temperature (RT). Blocking buffer solution used for detection of non phosphorylated protein contained 5% non-fat dry milk in 1×-TBS-T (50 mM Tris-HCl pH 7.4, 137 mM NaCl2, 2.7 mM KCl, 0.1% Tween). For detection of phosphorylated protein, blocking buffer solution contained 5% Bovine Serum Albumin in 1×TBS-T. Membranes were probed with primary antibody (various dilutions depending on the epitope—see below) in blocking buffer overnight at 4° C. on a rocker. Membranes were subsequently washed with 1×TBS-T and probed with secondary antibody in blocking buffer for forty-five minutes using a 1:25,000 dilution of HRP-goat anti-mouse or HRP-goat anti-rabbit, depending on the species (mouse or rabbit) in which the primary antibodies were raised. Following incubation with secondary antibody, membranes were washed in 1×TBS-T and developed with Super Signal West Femto Maximum Sensitivity Substrate Kit (Promega, Madison, Wis.) and imaged electronically. Protein band signal unsaturation was verified before any further analysis of multiple forms of tau. To test multiple primary antibodies, membranes were stripped for 15 minutes at RT using ReBlot Plus Mild Antibody Stripping Solution (Millipore, Millerica, Mass.). Membranes were then washed for 5 minutes in 1×TBS-T and blocked for one hour in 5% blocking solution at RT. For verification of protein loading, membranes were reprobed overnight at 4° C. with an anti-Tubulin primary antibody (1:1000 dilution). Primary antibodies used for detection included anti-tau (1:2000 dilution), 12E8 tau (1:7500 dilution), pT231 tau (1:1000 dilution), pS396 tau (1:5000 dilution), and anti-DYRK1A (1:500 dilution).

Compound Treatments:

Cells undergoing any treatment, including β-carboline derivative dosing and siRNA treatment, were maintained in Dulbecco's Modified Eagle Medium supplemented with 10% fetal bovine serum and 2 mM L-Glutamine. Viability assays were performed using a 96-well pate format. Metabolic activity was measured 12 hours after the addition of 10% alamar Blue directly to attached cells in full medium. This assay was based on the ability of metabolically active cells to convert alamar Blue reagent into a fluorescent signal proportional to innate metabolic activity. Once the ideal IC50 value for viability was identified, effects on multiple forms of tau were investigated after treating with the β-carbolines indicated in Table I using the larger 6 well plate format. For both the viability assay and cell culture tau assays, cells were treated with freshly made drug every 24 hours for 4 days. For cell culture tau assays, protein lysates were prepared after 96 hours of treatment. All compounds were solubilized in dimethylsulfoxide (DMSO), diluted in growth medium to their respective 0.01 μM, 0.1 μM, 1 μM and 10 μM final working dilutions and added directly to cultured cells. The final DMSO percentage in culture for all compounds and concentrations tested was 0.1%. All treatment conditions were compared to their respective controls which contained DMSO at 0.1% in growth medium.

In Vitro Kinase Assay:

Evaluation of DYRK1A kinase activity was determine by incubating 0.08 μg of recombinant human DYRK1A protein (Invitrogen, Carlsbad, Calif.) with 0.15 ug of 4R2N recombinant human tau (SignalChem, Richmond, Canada) in 1× kinase buffer (25 mM Tris-HCl (pH 7.5), 5 mM beta-glycerophosphate, 2 mM dithiothreitol (DTT), 0.1 mM Na3VO4, 10 mM MgCl2—Cell Signal) and 1 mM ATP in a final volume of 20 ul for 30 minutes at 30° C. For testing the effects of the β-carboline derivatives, recombinant human DYRK1A was pretreated with compounds for 10 minutes prior to the addition of kinase buffer, ATP, and recombinant human tau. The reaction was inactivated upon addition of 1× Novex LDS sample buffer and Novex sample reducing reagent, 50 mM DTT, followed immediately by heating for 10 minutes at 95 C. Phosphorylated tau was resolved using 7% Tris Acetate gels and detected by Western analysis. Westerns were probed for Phospho-tau S396 at 1:5,000 dilution and a secondary of Goat anti-Rabbit HRP (Jackson ImmunoResearch Labs, West Grove, Pa.) at 1:50,000 in 5% BSA. Membranes were stripped as above and reprobed with rabbit anti Human Total Tau at 1:15,000 dilution and a secondary of Goat anti-Rabbit HRP at 1:100,000 dilution in 5% milk.

Results:

Reduced DYRK1A Expression Affects Tau Phosphorylation at Multiple Sites

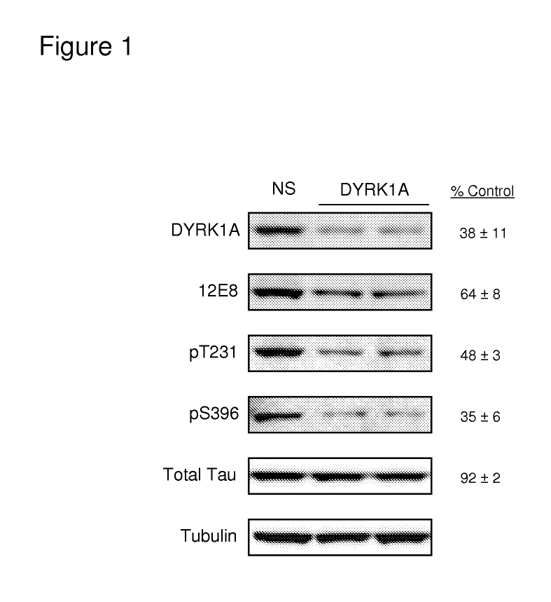

H4 neuroglioma cells that overexpress 4R0N (four repeat tau) were transfected with siRNA specific for DYRK1A. Silencing of DYRK1A was confirmed with anti-DYRK1A antibody (FIG. 1 top panel). It was found in the present invention that RNAi-mediated silencing of DYRK1A expression simultaneously affects multiple additional AD-relevant tau phosphorylation sites, including threonine 231 (T231) and serine 396 (S396) (FIG. 1). The reduction of DYRK1A expression to 38% of control leads to pT231 and pS396 tau expression that is 48% and 55% of control nonsilencing siRNA, respectively. The reduction of Tau 12E8 epitope (S262 and S356) was not as much as T231 and S396 sites. In other report, DYRK1A was found to be involved in the phosphorylation of tau S262, as well as sites S404, T212, S202.

The High Affinity DYRK1A Inhibitor, Harmine, Affects Tau Phosphorylation on Multiple Sites

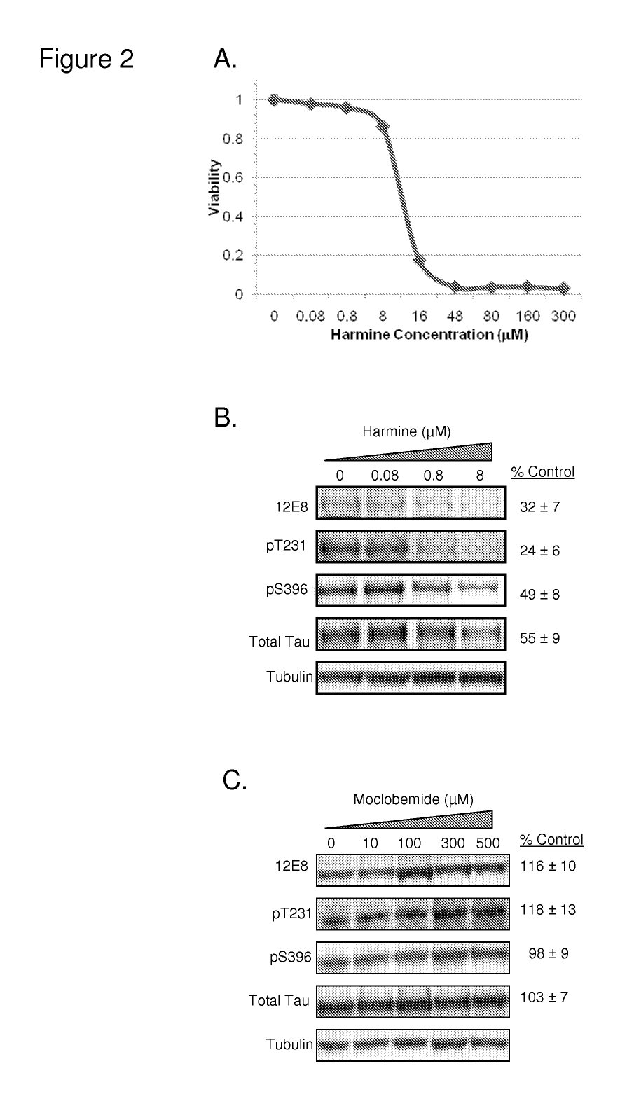

In the present invention, Harmine was tested for affects on tau phosphorylation in the H4 neuroglioma cell line. The toxicity profile for harmine (FIG. 2A) was first determined. Results of increasing concentrations of harmine showed that 12 μM resulted in 50% cell viability. Based on this toxicity profile, doses of 80 nM, 800 nM and 8 μM were selected for the tau phosphorylation assays. Harmine reduced the expression of each phospho-tau species tested, including 12E8 (pS262/pS356), pT231, and pS396 (FIG. 2B). However, harmine at 0.8 μM and 8 μM also has been shown to reduce the levels of total tau protein consistent with the reductions detected with the various phospho-tau antibodies.

Harmine's Effect on Tau does not Result from MAO-A Inhibition

Harmine has also been reported to be a selective inhibitor of monoamine oxidase (MAO-A). It was unknown whether MAO-A could affect tau phosphorylation, or whether the inhibition of MAO-A by harmine could affect tau phosphorylation, a selective antagonist, moclobemide was therefore tested in the present invention. Moclobemide has a reported IC₅₀ against MAO-A of 3.9 μM. Results showed that this MAO-A antagonist did not reduce levels of either total tau or of specific phosphorylated forms of tau protein at doses up to 500 μM (FIG. 2C). These results suggest that the effects of harmine on tau do not result from MAO-A inhibition.

Additional β-Carboline Alkaloid Derivatives Alter the Expression of Multiple Tau Species

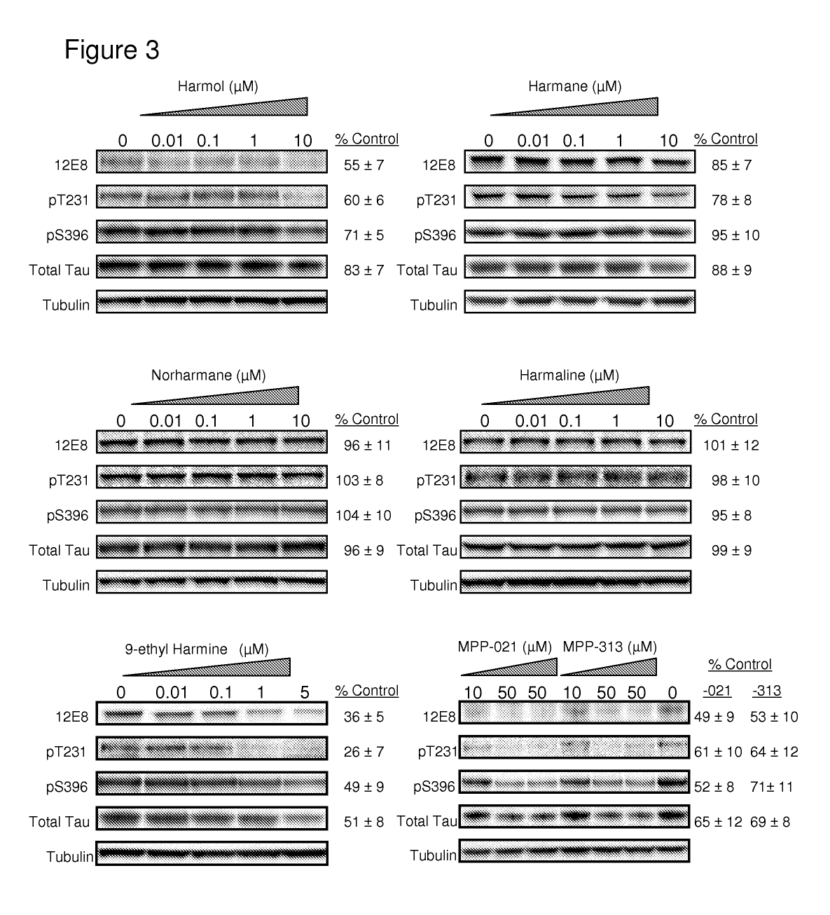

Based on results for harmine, additional β-carboline derivatives, including harmol, harmane, harmaline, norharmane, and 9-ethylharmine were tested (See Table 1 for compound structure). Harmol, harmane, norharmane, 9-ethylharmine are fully aromatic β-carboline compounds, whereas harmaline is a dihydro-derivative. Toxicity assays for of each compound in the H4 neuroglioma cell line were performed, and the IC₅₀ value is as follows: harmine 12 μM, harmol 18 μM, harmane 32 μM, harmaline 56 μM, norharmane 95 μM, and 9-ethylharmine 9 μM. Each of the above compounds was then tested for effects on phospho-tau and total tau expression (FIG. 3). A positive correlation between the toxicity of each compound and the sensitivity with which each compound reduced total tau levels and the levels of phosphorylated forms of tau was shown. For toxicity, the rank order of the compounds was 9-ethylharmine>harmine>harmol>harmane>harmaline>norharmane. In terms of the sensitivity with which each compound reduced tau levels, only 9-ethylharmine, harmine, and harmol showed significant effects in reducing total tau and phosphorylated tau levels at a dose of 10 μM. The 9-ethylharmine and harmine compound treatments showed significant reductions at 1 μM and 0.8 μM, respectively. These lower doses have no detectable effect on the viability of the cells. Reducing tau levels beyond 50% of the control levels, as occurs at higher concentrations, leads to significant cellular toxicity rather than the alternative of the observed reductions in tau resulting from general drug-induced toxicity. 9-ethylharmine clearly showed the most potent effect in this assay, significantly reducing total and phospho tau levels at a 1 μM concentration.

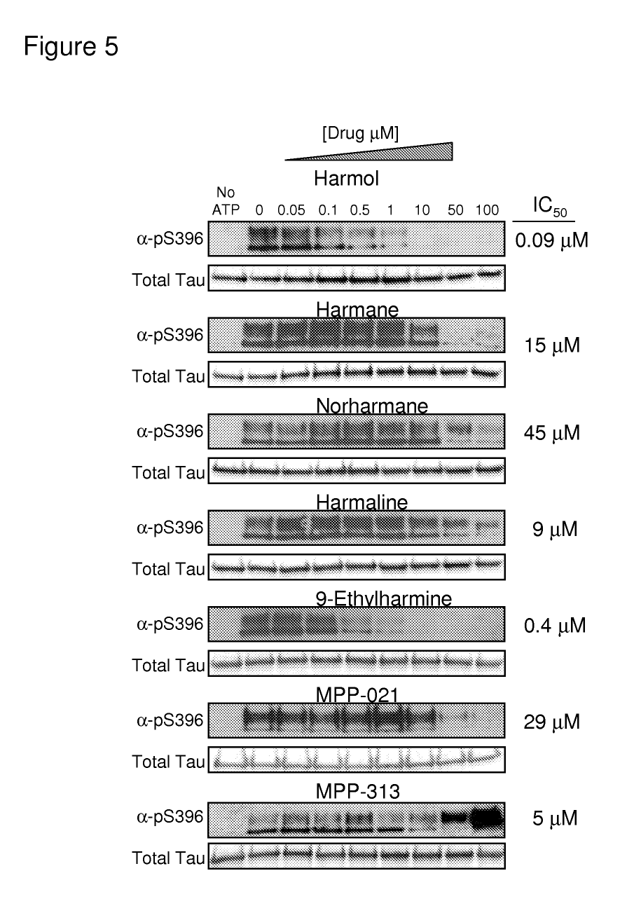

Modifications to certain structural components of the β-carboline ring structure significantly affected the ability of these compounds to inhibit tau phosphorylation. Harmaline is a beta-carboline derivative lacking the C3-C4 double bond. A comparison of results for harmaline and harmine indicate that a fully aromatic ring structure provides higher affinity for tau inhibition and toxicity (see Table I, FIG. 2C, FIG. 3 and FIG. 5). By comparing harmine and harmol to harmane, certain modifications to carbon 7 increased toxicity and effects on tau phosphorylation were demonstrated. For example, an —H at this carbon 7 position (harmane) had the lowest affinity. An —OH group (harmol) had the highest affinity in vitro, but reduced affinity in cell lines relative to the —OCH₃ group of harmine. In a comparison of norharmane to harmane, it appeared that the methyl group on carbon 1 (harmane) was important for the observed tau effects and toxicity. Lastly, comparing 9-ethylharmine to harmine, the addition of an ethyl group to N-9 increased the effects of harmine on tau and increased toxicity. Considering harmine's inhibition effect on MAO-A, the fact that 9-ethyl harmine is effective at a much lower dosage than harmine increases its value as a treatment candidate for AD related short-memory loss.

Harmine and Other β-Carboline Alkaloids Inhibit the Direct Phosphorylation of Tau by DYRK1A

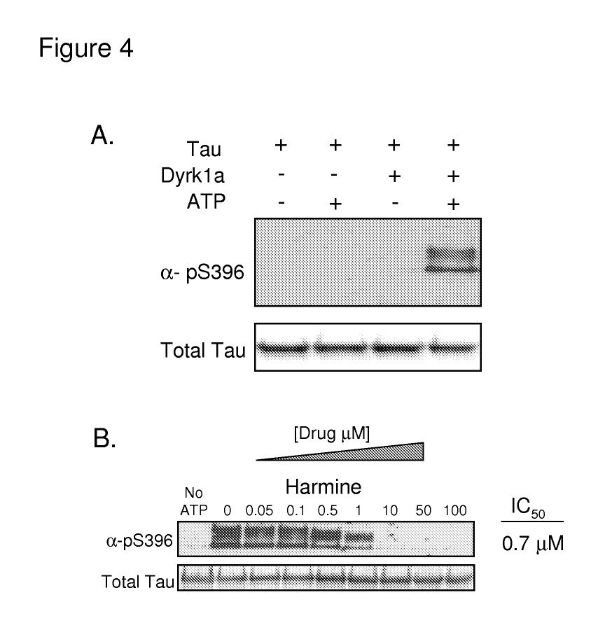

An in vitro phosphorylation assay with recombinant DYRK1A and tau proteins indicated that DYRK1A could directly phosphorylate tau protein (FIG. 4A). Phosphorylation of tau protein occurred only in the presence of tau protein, DYRK1A protein, and ATP. A doublet of pS396 phosphorylated tau (α-pS96) was observed. This pS396 tau phosphorylation was potently inhibited by harmine with an IC₅₀ of 0.7 μM (FIG. 4B).

Referring now to FIG. 5, IC₅₀ values for each compound for the inhibition of DYRK1A dependent tau phosphorylation at serine 396 are indicated. These results reflect the rank ordered affinities for each compound that were obtained in the cell based tau phosphorylation assays and the toxicity assays (Table 1, FIG. 2B and FIG. 3), with one exception. Harmol was the most potent inhibitor in this in vitro phosphorylation assay with an IC₅₀ of 90 nM, followed by 9-ethyl harmine (400 nM) and harmine (700 nM). In comparison, harmol was the third ranked compound in both the toxicity and cell-based tau assay. Reasons for this slight disconnect are unclear, but could be related to differential cellular metabolism of the free hydroxyl group on carbon 7 of harmol.

The addition of an ethyl group to N-9 of harmine reduced the IC₅₀ nearly 2-fold, suggesting that additional modifications on this nitrogen might increase the affinity of harmine for DYRK1A more substantially. Harmane, norharmane, and harmaline were more than an order of magnitude lower in affinity than harmine, which is consistent with the relatively muted effects of these compounds in the cell-based tau assay (FIG. 3).

### Example 2

This example demonstrates that Harmine significantly enhances hippocampal-dependent working memory.

Materials and Methods:

Subjects:

Twenty-six 17 month-old Fischer-344 male rats raised at the National Institute on Aging colony at Harlan Laboratories (Indianapolis, Ind.) were used in the study. After arrival, rats were pair-housed, had food and water ad-lib, and were maintained on a 12-h light/dark cycle. Procedures were approved by the Institutional Animal Care and Use Committee, and adhered to National Institutes of Health standards.

Experimental Design and Drug Treatments:

Rats were randomly divided into three treatment groups (n at start of study, m included in final behavioral analyses): vehicle (10μ, 10), low-Harmine 1 mg/kg (10μ, 10), or high-Harmine 5 mg/kg (10, 6). Nine days after arrival, animals started receiving daily subcutaneous injections at a volume of 1 ml/kg. Harmine (Acros Organics, Harmine hydrochloride hydrate 98%) was prepared daily, and dissolved in saline (NaCl 0.9%). Behavioral testing began after the second injection day, testing commenced approximately 30-45 minutes after injections and lasted for 6-8 hours. Animals were assigned semi-randomly to one of three testing squads of 10 animals each balanced with respect to treatment group.

Delayed-Match-to-Sample Asymmetrical 3-Choice Task:

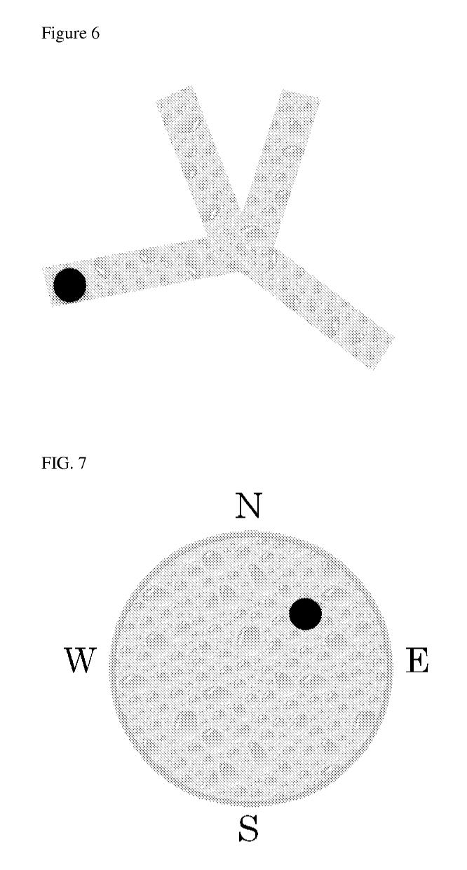

Spatial working memory and short-term memory retention were evaluated using a win-stay water-escape DMS asymmetrical place-learning task. The maze was an asymmetrical, four-arm apparatus (each arm 38.1×12.7 cm), filled with opaque, room temperature water containing a submerged platform (10 cm diameter) in one of the 4 arms (FIG. 6). This task was identical to the win-stay DMS plus maze, with the exception of the asymmetrical arm configuration. Animals were released into a different start arm at the beginning of each trial, varying semi-randomly such that the animals were released from each of the three non-platformed arms twice within a day of testing. The platform remained in the same location within a day, but changed location across days. Animals received 6 trials/day with 90 seconds to locate the platform, 15 seconds on the platform and a 30 second inter-trial-interval in a heated cage for nine days. Trial 1 was the information trial, trial 2 was the working memory trial and trials 3-6 were considered recent memory trials. Entry into any non-platformed arm was counted as an error. An arm entry was counted when the tip of a rat's snout reached a mark on the outside of the arm (not visible from the inside of the maze; 11 cm into the arm).

Morris Water Maze:

Spatial reference memory was evaluated using the Morris Maze. The apparatus was a round tub (188 cm diameter) filled with opaque room temperature water containing a submerged platform (10 cm diameter) (FIG. 7). The platform remained in a fixed location across days and trials, testing spatial reference memory. Testing consisted of 6 trials/day for 3 days. Animals were dropped off at different starting points (north, south, east or west) for each trial, varying semi-randomly. Animals had 60 seconds to locate the platform where they remained for 15 seconds before being placed back into a heated cage awaiting the next trial. The inter-trial-interval was approximately 5-8 minutes. To evaluate whether animals spatially localized the platform, a probe trial was given on trial seven on the third day of testing, during which the platform was removed and animals were given 60 seconds to swim freely in the maze. A video camera and tracking system tracked and measured each rat's swim pathway.

Visible Platform Task:

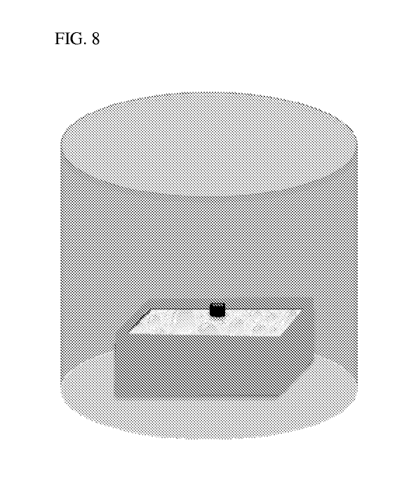

Four days after MM testing, motor and visual competence were evaluated using the visible platform task. This was an adaptation of the cue-navigation version of the spatial MM task previously used to dissociate visual and motor acuity from place memory. This task was ideal due to its similarity to other spatial water-maze tasks with respect to motor and visual requirements, differing only in that animals are not required to associate the location of the platform with distal cues. The apparatus was a rectangular tub (39×23 in) filled with clear room temperature water. A black platform (10 cm wide) was positioned 1.5″ above the surface of the water following previously published methods. Opaque curtains surrounded the maze to block distal cues (FIG. 8). Animals were given 6 consecutive trials in one day. The drop off location remained the same across trials, and the platform location for each trial varied semi-randomly across three locations. Each rat had 90 seconds to locate the platform, where it remained for 15 seconds before being placed back into its heated cage awaiting the next trial. The inter-trial-interval was approximately 5-8 minutes.

Statistical Analyses:

In an initial analysis, Harmine-low and Harmine-high groups were compared. There were no statistical differences between the two harmine-treated groups for any measure on the DMS and MM tasks. Therefore, for final analyses both harmine-treatment groups were combined so that the final treatment groups were as follows (n in parentheses): Vehicle (10), Harmine (16). DMS testing was divided into 4 testing blocks consisting of 2 days each. DMS data were analyzed with an omnibus ANOVA with treatment as the between groups variable and total number of errors for each trial as repeated measures. MM data were analyzed with an omnibus ANOVA with treatment as the between groups variable and swim distance (cm) to the platform as repeated measures. For MM probe trial data, percent distance in the previously platformed (target) quadrant was compared with the diagonally opposite quadrant using repeated-measures (quadrant) ANOVA. Theoretically, rats that spatially localized the platform should spend a greater percent distance in the target vs. opposite quadrant. Visible platform data were further analyzed using a regression analysis with testing squad (1-3) as the predictor and mean escape latency across trials 1-6 as the criterion. All analyses were two-tailed, alpha <0.05.

Results:

Harmine Enhances Working and Short-Term Memory

Harmine treatment enhanced working and short-term memory on the DMS task on the lattermost portion of testing. However, harmine also elicited obvious motoric effects. Only Harmine-high animals that were tested more than 2 hours after injections were included in these analyses. Many of these animals also showed motor deficits earlier in the day, closer in time to injections, which were resolved by the time they were tested in the water mazes. Therefore it was important to use selective visible platform task to verify motoric and visual competence in strenuous tasks such as water maze testing to avoid distractions from evaluating cognitive performance after harmine treatment.

Visible Platform Task:

During testing it was noted that several subjects had motor difficulties impacting swim ability. Given that these motor challenges would likely impact interpretation of performance, the visible platform data underwent the initial series of analyses to gain insight into which subject had the procedural capability to perform the task.

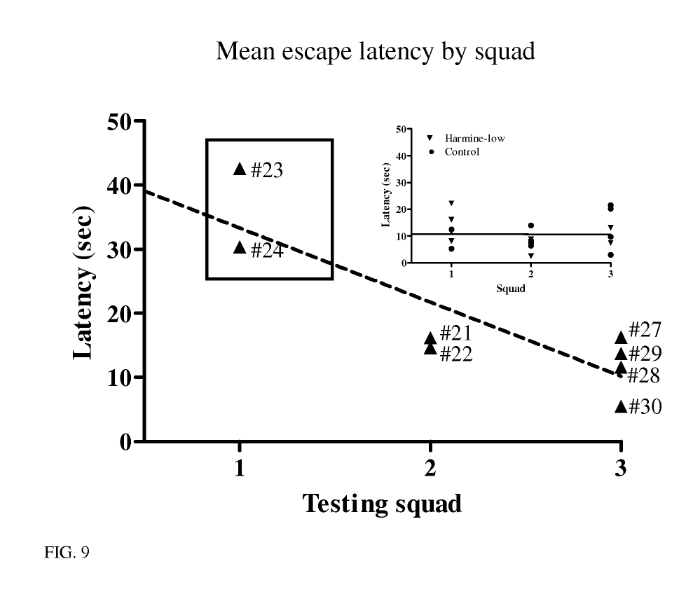

The visible platform task was used to confirm that animals have the ability to perform the procedural components of water-escape maze testing, including the visual and motoric capacities necessary to swim towards and climb onto a platform. Two Harmine-high animals were repeatedly unable to perform both cognitive tasks due to obvious motoric difficulties, and had to be removed from the maze on multiple occasions. These animals were removed from the study before completion of the second maze task, and were therefore not tested on the visible platform task. FIG. 9 shows average swim time across all 6 trials on the visible platform task for each subject in order of testing squad. For Harmine-high treated animals, testing squad (1-3) was a significant predictor of mean escape latency (β=−11.567, SE=2.751, p=0.006, R²=0.75) with mean escape latency decreasing by an average of roughly 11.6 seconds with each squad tested and testing squad accounting for 75% of the total variance in mean escape latency (FIG. 9). Testing squad was not a significant predictor of mean escape latency in the Harmine-low (β=−2.968, SE=2.255, p=0.224 NS, R²=0.18) or Control (β=2.696, SE=2.619, p=0.333 NS, R²=0.12) groups. To simplify presentation, Harmine-low and Control groups are presented together (β=0.08, SE=1.702, p=0.96 NS, R²<0.01) (FIG. 9). Following this analysis, any animals that had a mean swim time exceeding two standard deviations of the mean swim time for vehicle-treated animals across all six trials were identified. Two animals from the Harmine-high treatment group (#23 and #24) were identified by these criteria and excluded from the analysis. Once these animals were excluded, testing squad was no longer a significant predictor of mean escape latency within the Harmine-high treated animals (β=−3.659, SE=3.485, p=0.35 NS, R²=0.21, data not shown). Therefore, these two animals (#23 and #24) were designated as lacking the motor and/or visual competencies required to perform a water-escape task and were excluded from all other statistical analyses for DMS and MM to avoid a potential confound with our cognitive measures, bringing the final number of Harmine-high treated subjects to six.

All 4 of the Harmine-high treated animals that were ultimately excluded from analyses were tested at the beginning of the day, in close temporal proximity to injections. The two animals that were physically unable to complete testing were the first two animals to be tested each day and both showed persistent and severe motor problems, such that they were unlikely to survive behavioral testing had it continued. It is important to note that these 4 animals exclusively comprised the subset of Harmine-high treated animals in the first testing squad and all were tested daily within 90 minutes of injection. The observed side-effects were not limited to these 4 animals, in fact most Harmine-high treated animals demonstrated similar impairments lasting roughly 1-2 hours after injections, followed by qualitatively normal behavior until the next round of injections. The observed motor difficulties were sufficient to hinder the Harmine-high animals' abilities to walk or stand, including standing on the just-located platform for animals whose behavioral testing coincided with the period of side effects.

DMS Asymmetrical 3-Choice Task:

There were no main effects of Harmine treatment for testing blocks 1, 2 or 3. For testing block 4, the lattermost portion of testing, there was a main effect of Harmine treatment for trials 2-6 (F_{1,24}=5.036, P=0.03), with animals treated with Harmine making fewer total errors relative to animals treated with saline, indicating that Harmine treatment enhanced working memory (FIG. 10). Animals receiving harmine made significantly fewer errors locating the submerged platform in this test. Therefore, harmine significantly enhanced hippocampal-dependent working memory.

Morris Water Maze:

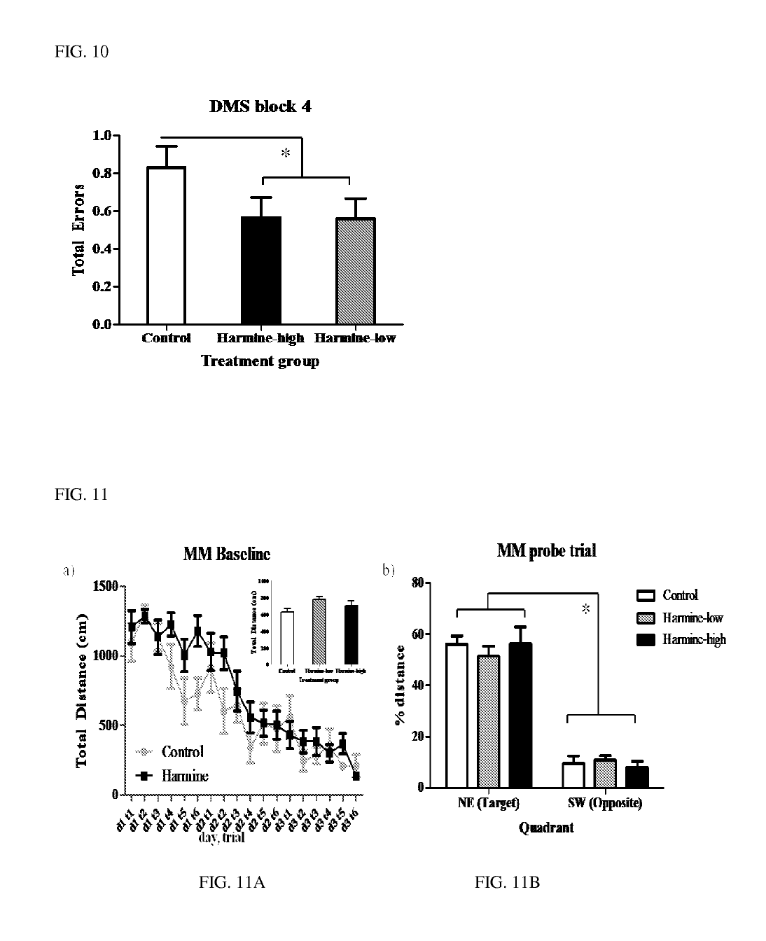

There were no Harmine treatment main effects for distance on days 1-3 (FIG. 11A). For the probe trial, a higher percent distance was spent in the previously platformed vs. the opposite quadrant (quadrant main effect: F_{1,24}=149.187; P<0.0001). This was in the absence of a Treatment×Quadrant interaction, indicating that all groups localized to the previously platformed quadrant (FIG. 11B). These results indicated no significant effect of harmine on spatial reference memory at the doses tested.

What is claimed is:

1. A pharmaceutical composition, the composition comprising at least one pharmaceutically acceptable carrier and at least one compound selected from the group consisting of 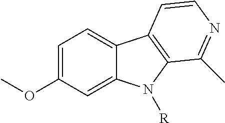
wherein R is selected from the group consisting of H, halo, —C₁-C₆ alkyl, aryl, —C₃-C₇ cycloalkyl, and -3- to 10-membered heterocycle, harmine, harmol, harmane, norharmane, harmaline, and 9-ethyl harmine.

2. The pharmaceutical composition of claim 1, wherein the composition comprises 9-ethyl harmine and at least one pharmaceutically acceptable carrier.

3. The pharmaceutical composition of claim 1, wherein the composition comprises harmol and at least one pharmaceutically acceptable carrier.

4. The pharmaceutical composition of claim 1, wherein the composition comprises harmane and at least one pharmaceutically acceptable carrier.

5. The pharmaceutical composition of claim 1, wherein the composition comprises harmine and at least one pharmaceutically acceptable carrier.

6. The pharmaceutical composition of claim 1, wherein the composition comprises at least one pharmaceutically acceptable carrier and at least one compound selected from the group consisting of 9-ethyl harmine, harmol, harmane and harmine.

7. A method of treating a disorder characterized by phosphorylation of a serine or threonine residue of SEQ ID NO. 1 comprising the step of:
administering a therapeutically effective dose of a pharmaceutical composition to a subject, wherein said pharmaceutical composition comprises a compound selected from the group consisting of 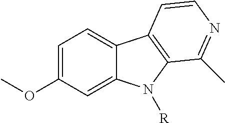
wherein R is selected from the group consisting of H, halo, —C₁-C₆ alkyl, aryl, —C₃-C₇ cycloalkyl, and -3- to 10-membered heterocycle, harmine, harmol, harmane, norharmane, harmaline, and 9-ethyl harmine.

8. The method of claim 7, wherein the disorder is Alzheimer's disease.

9. The method of claim 7, wherein the disorder is Down's syndrome.

10. The method of claim 7, wherein the serine or threonine residue is selected from the group consisting of serine-262, threonine-231, and serine-396.

11. The method of claim 7, wherein the pharmaceutical composition comprises 9-ethyl harmine and at least one pharmaceutically acceptable carrier.

12. The method of claim 7, wherein the composition comprises harmol and at least one pharmaceutically acceptable carrier.

13. The method of claim 7, wherein the composition comprises harmane and at least one pharmaceutically acceptable carrier.

14. The method of claim 7, wherein the composition comprises harmine and at least one pharmaceutically acceptable carrier.

15. The method of claim 7, wherein the composition comprises at least one pharmaceutically acceptable carrier and at least one compound selected from the group consisting of 9-ethyl harmine, harmol, harmane and harmine.

16. A method of enhancing the working memory of a subject comprising the step of:
administering a therapeutically active dose of a pharmaceutical composition to a subject, wherein said pharmaceutical composition comprises a compound selected from the group consisting of 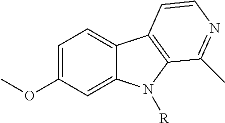
wherein R is selected from the group consisting of H, halo, —C₁-C₆ alkyl, aryl, —C₃-C₇ cycloalkyl, and -3- to 10-membered heterocycle, harmine, harmol, harmane, norharmane, harmaline, and 9-ethyl harmine.

17. The method of claim 16, wherein the pharmaceutical composition comprises 9-ethyl harmine and at least one pharmaceutically acceptable carrier.

18. The method of claim 16, wherein the composition comprises harmol and at least one pharmaceutically acceptable carrier.

19. The method of claim 16, wherein the composition comprises harmane and at least one pharmaceutically acceptable carrier.

20. The method of claim 16, wherein the composition comprises harmine and at least one pharmaceutically acceptable carrier.

21. The method of claim 16, wherein the composition comprises at least one pharmaceutically acceptable carrier and at least one compound selected from the group consisting of 9-ethyl harmine, harmol, harmane and harmine.

22. The method of claim 16, wherein the subject has Alzheimer's disease.

23. The method of claim 16, wherein the subject has Down's syndrome.


SEQUENCE LISTING (1 sequence)

SEQ ID NO: 1 - protein, 758 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL